(19) United States
(12) Patent Application Publication
(10) Pub. No.: US 2006/0217727 A1
(43) Pub. Date: Sep. 28, 2006

(54) Bone screw

(76) Inventors: Chad Munro, Nabou (CA); Peter Senn, Waldenburg (CH)

Correspondence Address: JONES DAY, 222 EAST 41ST STREET, NEW YORK, NY, 10017-6702, US

(21) Appl. No.: 10/518,867

(22) PCT Filed: Apr. 3, 2003
(86) PCT No.: PCT/CH03/00222

§ 371 Date: Feb. 15, 2006

(30) Foreign Application Priority Data
Jun. 21, 2002 (CH) 02/00337

(51) Int. Cl. A61B 17/58

(52) U.S. Cl. 606/73

(57) ABSTRACT

A bone screw having a threaded shaft 15 which has a thread having an outer diameter D_{A}, a frontal thread end and a thread profile. The thread profile has a frontal thread flank which is directed towards the frontal thread end 4, a rear thread flank 18 and a flank angle δ. The thread is multipath, preferably a double path thread where each thread path has a blade-type element with a cutting edge on the frontal thread end. The bone screw 1 also has a longitudinal axis 2. For accommodating a guide wire, the bone screw 1 is provided with a central borehole 9, which is coaxial with its longitudinal axis 2.

### TECHNICAL FIELD

The present invention generally relates to a bone screw.

### BACKGROUND OF THE INVENTION

A screw with a self-cutting threaded section for plastic materials, which includes, at the front end of the threaded section, a knife-like element, is known in the art. (See U.S. Pat. No. 6,158,939 to Grossberndt) A disadvantage of the Grossberndt screw is that the threaded section includes only one thread. Because of the single, knife-like element, the load on the bone screw while cutting the threaded section is asymmetric and a radial force is exerted on the bone screw which leads to a lateral migration of the tip of the screw. This can lead to a wobbly, eccentric cutting process that results in a cross sectional enlargement in the bone material. The threads, cut in this manner into the bone material, are larger than the thread profile of the bone screw, as a result, the hold of the bone screw in the bone material is decreased.

### SUMMARY OF THE INVENTION

The present invention provides a remedy for the above-discussed disadvantage. An object of the present invention to provide a bone screw, the external thread of which can be cut concentrically with the axis of the screw into the soft bone material, so that it is held in the bone material over the whole circumference by means of a press fit.

The present invention accomplishes the objectives set out above with a bone screw having a longitudinal axis. The bone screw comprises a threaded shaft which includes a threaded section with an external diameter D_{A}, a frontal thread end of the threaded section, and a thread profiler. The thread profile has a front threaded flank directed towards the frontal thread end of the threaded section, a rear threaded flank and a flank angle β, and the threaded section has a multiple thread where each thread of the threaded section at the frontal thread end of the threaded section has a knife-like element with a cutting edge.

Advantages achieved by the present invention are that the knife-like element, disposed at each front end of the multiple thread, permits a symmetrical distribution of the forces during the cutting of the thread, radial pressing of the bone material takes place concentrically, so that the danger of the formation of micro or macro fractures in the bone material can be confined appreciably. A desirable, radial pre-tensioning in the bone material is maintained, at least partially, so that there can be no loosening and, resulting therefrom, absorption of the bone material. Another advantage realized by the present invention is that larger projection surface crossways to the longitudinal axis of the screw and, with that, larger working surface crossways to the longitudinal axis of the screw can be achieved.

In another embodiment, the threaded section is constructed as a double thread.

In a different embodiment, a sector angle of the knife-like element is between 45° and 200° of a thread and preferably between 55° and 200° of a thread. This configuration of the knife-like element has the advantage that it does not have an excessively large cutting angle for use in a bone and the threaded profile is weakened only on a portion of the first thread.

In still another embodiment, the cutting edge of the knife-like element is constructed in such a manner, that it encloses a sector angle a of at least 45° and preferably of at least 55° of a thread. The advantages achieved by such a configuration are that the external threaded section has knife-like elements with flank angles which are smaller than the flank angles of the threaded section, and there are cutting edges only on the first thread so that the width is reduced only in this area. Due to this configuration of the front end of the threaded section, better retention can be achieved especially in the spongiosa of osteoporotic bones. This is of great importance especially for short bone screws which are used with osteoporotic, long bones, for example, with the osteoporotic proximal femur or with the osteoporotic proximal humerus. Due to the large sector angle of the cutting edge on the first thread, a cutting angle, suitable for bones, especially for the spongiosa of a bone, can be achieved.

In still another embodiment, the knife-like element is configured in such a manner, that it encloses an angle β between 10° and 60° and preferably between 15° and 35° with the urea thread flank. With this configuration, a sharp cutting edge may be produced at the knife-like element.

In yet another embodiment, the knife-like element is configured in such a manner, that the cutting edge is configured helically between the core and the tip of the thread.

In a further embodiment, the knife-like element is configured in such a manner that the cutting angle γ′ at the tip of the threaded section, that is, the angle between the tangent at the cutting edge and the tangent at the enveloping surface of the threaded section is between 20° and 60° and preferably between 35° and 50° at the tip of the threaded section. In the case of a spiral configuration of the cutting edge, the cutting angle γ′ varies radially. The cutting angle γ′ determines the cutting behavior of the knife-like element.

In yet another embodiment, the cutting edge at the knife-like element has a constant cutting angle γ′ between the core and tip of the threaded section.

In a further embodiment, the bone screw is configured in such a manner, that the front end of the threaded section coincides with the front end of the bone screw. The advantage of such an embodiment is that it can also be used for flat bone fragments.

Depending on the use, the front end of the bone screw may be convex or preferably spherical or also be provided with a conical facet.

Preferably, the knife-like element is sickle-shaped, the cutting edge being on the outer edge of the sickle-shaped, knife-like element.

Other objectives and advantages, in addition to those discussed above, will become apparent to those skilled in the art during the course of the description of the embodiments of the invention which follows. In the description, reference is made to accompanying drawings, which form a part thereof, and which illustrate examples of the invention. Such examples, however, are not exhaustive of the various embodiments of the invention, and therefore, reference is made to the claims that follow the description for determining the scope of the invention.

### BRIEF DESCRIPTION OF THE DRAWINGS

The bone screw is explained in even greater detail in the following exemplary drawings. The bone screw may be better understood by reference to the following drawings, wherein like references numerals represent like elements. The drawings are merely exemplary to illustrate the structure of the bone screw and the invention should not be limited to the embodiments shown.

FIG. 1 shows a longitudinal section of an embodiment of the inventive bone screw,

FIG. 2 shows a front view of the embodiment of the inventive bone screw shown in FIG. 1,

FIG. 3 shows a perspective view of the embodiment of the inventive bone screw shown in FIGS. 1 and 2,

FIG. 4 shows a side view of the embodiment of the inventive bone screw shown in FIGS. 1 and 2,

FIG. 5 shows a longitudinal section of another embodiment of the inventive bone screw

FIG. 6 shows a front view of the embodiment of the inventive bone screw shown in FIG. 5, and

FIG. 7 shows a longitudinal section through a further embodiment of the inventive bone screw.

### DESCRIPTION OF THE PREFERRED EMBODIMENTS

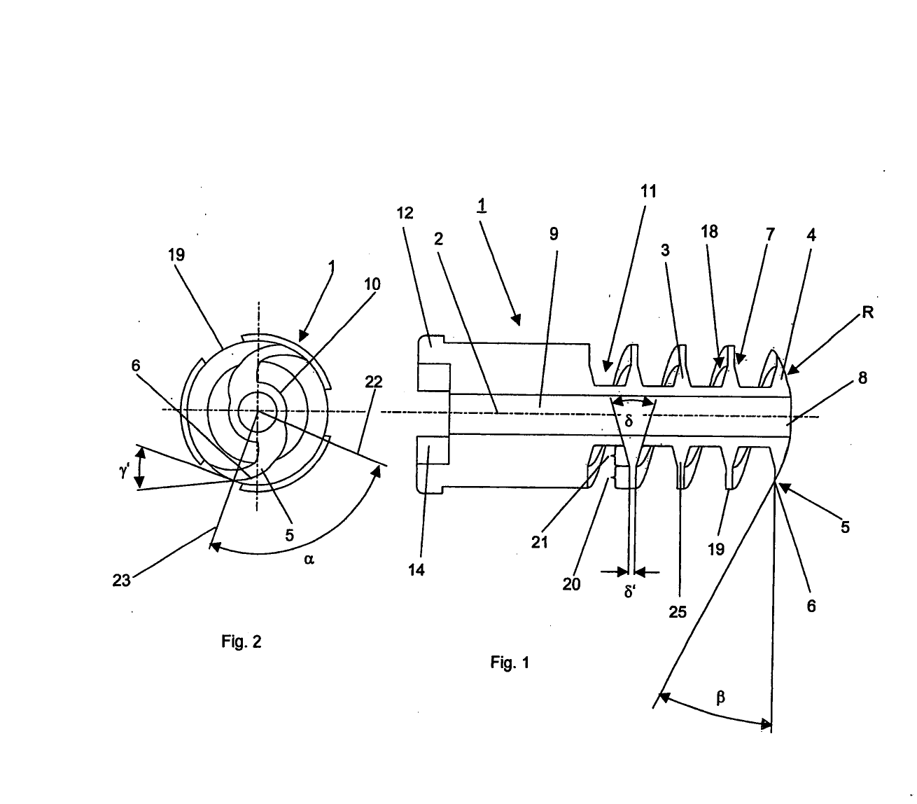

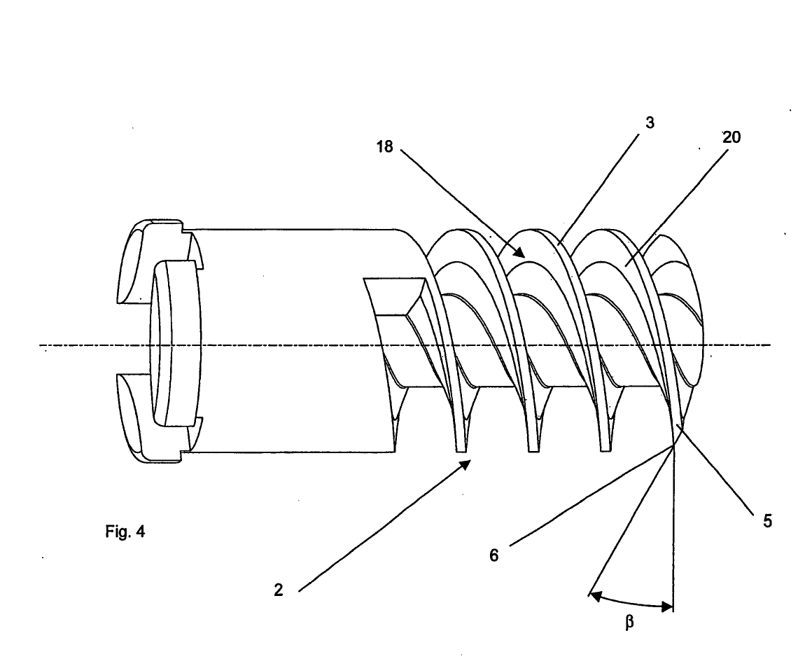

FIGS. 1 to 3 depict a preferred embodiment of the bone screw 1. The bone screw 1 comprises a threaded shaft 15. The threaded shaft 15 has a thread 3 having an outer diameter D_{A}, a frontal thread end 4 and a thread profile 11. The thread profile 11 includes a frontal thread flank 7 which is directed towards the frontal thread end 4, and a rear thread flank 18. The bone screw 1 has a longitudinal axis 2. For accommodating a guide wire, the bone screw 1 is provided with a central borehole 9, which is coaxial with its longitudinal axis 2.

In a preferred embodiment, the thread 3 is a multipath, preferably a double path thread (double thread) where each thread path of the thread 3 at the frontal thread end 4 comprises a blade-type (knife-like) element 5 that may be sickle-shaped with a convexly curved cutting edge 6 disposed between the core 10 and the tip 19 of the threaded section 3. At the tip 19 of the threaded section 3, the cutting edge 6 changes over with a local cutting angle γ′ into the enveloping surface of the threaded section 3. Since the cutting edge 6 has a spiral shape, the cutting angle γ′ varies between the tangent 16 at the cutting edge 6 and the tangent 17 of a circle, concentric with the longitudinal axis 2 between the core 10 and the tip 19 of the threaded section. The configuration of the knife-like element 5 is such, that the cutting edge 6 encloses a sector angle α between two radial straight lines 22, 23.

In one embodiment, the knife-like element 5 is configured in such a manner that the cutting angle γ′ at the tip of the threaded section 3, the angle between the tangent at the cutting edge and the tangent at the enveloping surface of the threaded section, is between 20° and 60° and preferably between 35° and 50°. In the case of a spiral configuration of the cutting edge, the cutting angle γ′ varies radially. The cutting angle γ′ determines the cutting behavior of the knife-like element.

In yet another embodiment, the cutting edge 6 at the knife-like element 5 has a constant cutting angle γ′ between the core and tip of the threaded section.

In one embodiment, the cutting edge 6 of the knife-like element 5 is constructed in such a manner, that it encloses a sector angle α of at least 45° and preferably of at least 55° of a thread.

In another embodiment, the sector angle α of the knife-like element 5 is between 45° and 200° of a thread and preferably between 55° and 200° of a thread. This configuration of the knife-like element has the advantage that it does not have an excessively large cutting angle for use in a bone and the threaded profile is weakened only on a portion of the first thread.

The front thread flank 7 and the rear thread flank 18 radially adjoin, forming at least two thread ridges 25, and have a peripheral, outer flank segment 20 and an inner flank segment 21. The two flank segments 20, 21 have different flank angles δ′ at the periphery of the thread and δ near the core 10, where δ is larger than δ′ and the flank angle δ′ of the outer flank segment 20 is 0° in this embodiment.

The bone screw 1 is configured such that the threaded section 3 extends up to the front end 8 of the bone screw 1 and, accordingly, the frontal thread end 4 coincides with the front end 8 of the bone screw 1. Furthermore, the front end 8 of the bone screw 1 is configured convexly in this embodiment. The convex, front end 8 may be spherical with a radius R so that the knife-like element 5 encloses an angle β, measured at the tip 19 of the threaded section with the rear threaded flank 18 and with the outer flank segment 20 forming the pointed cutting edge 6 at the tip 19 of the threaded section.

In one embodiment, the knife-like element 5 is configured in such a manner, that it encloses an angle β between 10° and 60° and preferably between 15° and 35° with the rear threaded flank. With this configuration a sharp cutting edge can be produced at the knife-like element.

In yet another embodiment, the knife-like element 5 is configured such that the cutting edge 6 is configured helically between the core and the tip of the thread.

At the rear end 12 of the bone screw 1, means 14 for accommodating a screwdriver, which is configured here as a hexagon socket, are disposed.

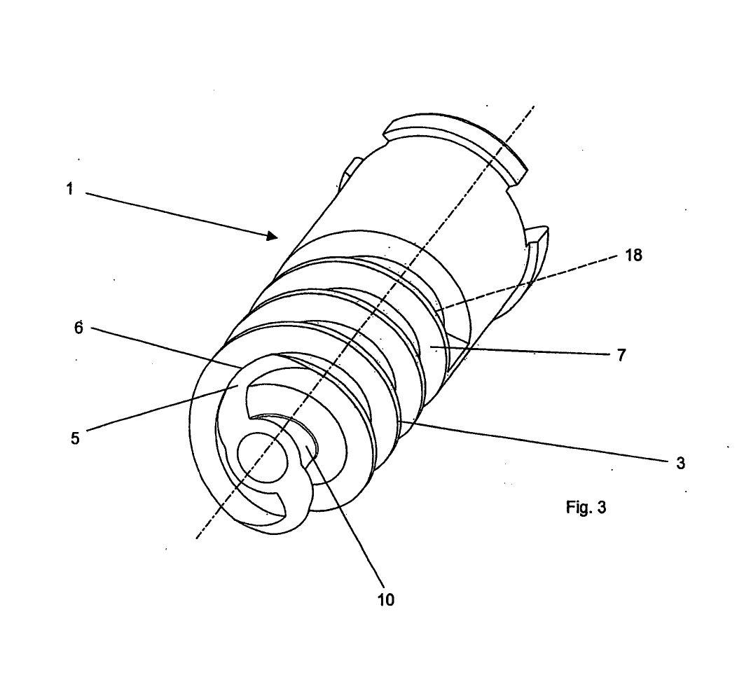

FIG. 4 shows a side view of the embodiment of the bone screw 1 shown in FIGS. 1 and 2. The angle β between the outer flank segment 20 of the rear threaded flank 18 and the knife-like element 5 is more readily identifiable.

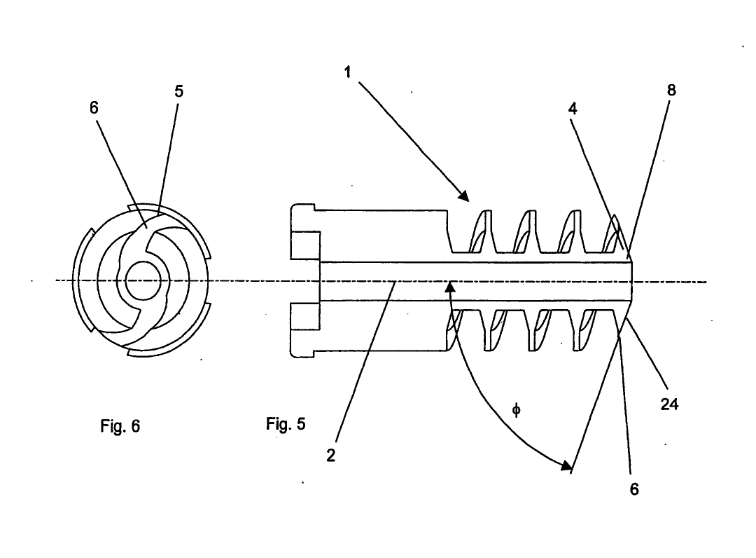

The embodiment of the bone screw 1, shown in FIGS. 5 and 6, differs from that shown in FIGS. 1 and 2 only in that the front end 8 of the bone screw 1 is not curved spherically. Instead the bone screw 1 of this embodiment is configured with a conical facet 24. The facet 24 encloses an angle φ with the longitudinal axis 2.

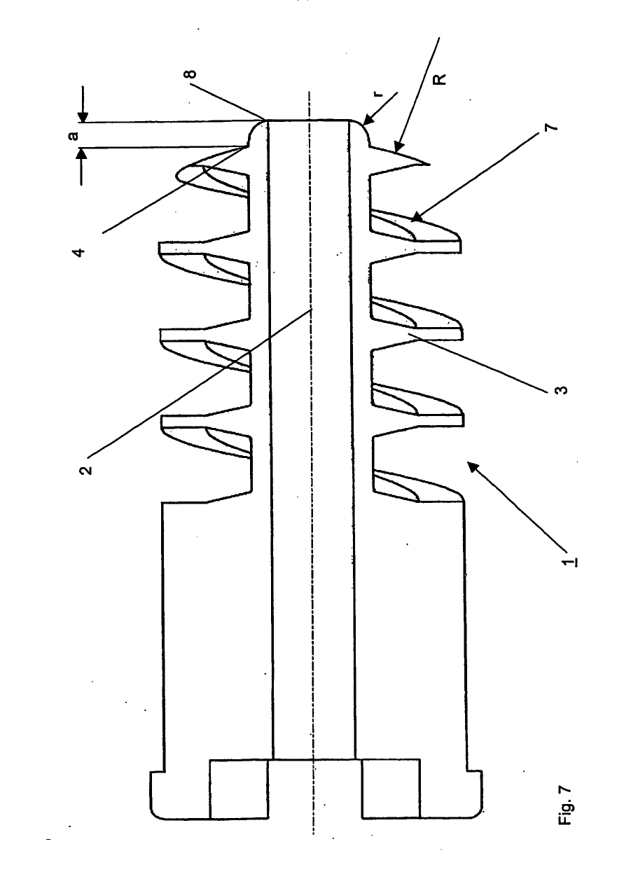

The embodiment of the bone screw 1, shown in FIG. 7, differs from that shown in FIGS. 1 and 2 only in that the frontal thread end 4 of the threaded section 3 is set back by a distance “a” from the front end 8 of the bone screw 1. Furthermore, the front flank 7 of the threaded section 3 is constructed spherically with a radius R at the frontal thread end 4. The front end 8 of the bone screw 1 is also rounded off and has a radius r.

Although the present invention has been described with reference to certain preferred embodiments, various modifications, alterations, and substitutions will be apparent to those skilled in the art without departing from the spirit and scope of the invention, as defined by the appended claims.

1. A bone screw having a longitudinal axis comprising:
a threaded shaft which includes a threaded section with an external diameter D_{A}, a frontal thread end of the threaded section, and a thread profile,
wherein the thread profile has a front threaded flank directed towards the frontal thread end (4) of the threaded section, a rear threaded flank and a flank angle β,
wherein the threaded section has a multiple thread, and each thread of the threaded section at the frontal thread end of the threaded section has a knife-like element with a cutting edge.

2. A bone screw according to claim 1, wherein the threaded section has a double thread.

3. A bone screw according to claim 1, wherein the cutting edge encloses a sector angle α of at least 45° and preferably of at least 55° of a thread.

4. A bone screw according to claim 1, wherein the cutting edge is curved convexly.

5. A bone screw according to claim 3, wherein the sector angle α is between 45° and 200° of a thread.

6. A bone screw according to claim 5, wherein the sector angle α is between 55° and 200° of a thread.

7. A bone screw according to claim 1, wherein the knife-like element encloses an angle β of between 10° and 60° with the rear threaded flank.

8. A bone screw according to claim 7, wherein the knife-like element encloses an angle β of between 15° and 35° with the rear threaded flank.

9. A bone screw according to claim 1, wherein the cutting edge between the core and a tip of the thread is constructed helically.

10. A bone screw according to claim 1, wherein the knife-like element has a cutting angle γ′ of between 20° and 60° at least at a tip of the threaded section.

11. A bone screw according to claim 10, wherein the knife-like element has a cutting angle γ′ of between 35° and 50° at least at the tip of the threaded section.

12. A bone screw according to claim 1, wherein the knife-like element has a constant cutting angle γ.

13. A bone screw according to claim 1, wherein the front end of the threaded section coincides with the frontal thread end of the bone screw.

14. A bone screw according to claim 13, wherein the front end is convex.

15. A bone screw according to claim 13, wherein the front end of the bone screw comprises a facet, which encloses an angle φ of between 30° and 90° with the longitudinal axis.

16. A bone screw according to claim 13, wherein the knife-like element, viewed from the front end of the bone screw, is constructed sickle-shaped with a cutting edge disposed on the outer edge.

17. A bone screw according to claim 1, wherein the threaded section comprises at least two thread ridges, which have a flank angle δ at the core of the threaded section and a flank angle δ′ at the periphery of the thread.

18. A bone screw according to claim 17, wherein the flank angle δ at the core of the threaded section is larger than the flank angle δ′ at the periphery of the thread.

19. (canceled)
